(19) United States
(12) Patent Application Publication
(10) Pub. No.: US 2017/0096620 A1
(43) Pub. Date: Apr. 6, 2017

(54) Polypeptides Having Phospholipase C Activity and Polynucleotides Encoding Same

(71) Applicant: Novozymes A/S, Bagsvaerd (DK)

(72) Inventors: Tianqi Sun, Beijing (CN); Ming Li, Beijing (CN); Kim Borch, Birkerod (DK); Allan Noergaard, Lyngby (DK); Jesper Brask, Vaerlose (DK); Marianne Linde Damstrup, Gentofte (DK); Hanna Maria Lilbaek, Copenhagen (DK)

(73) Assignee: Novozymes A/S, Bagsvaerd (DK)

(21) Appl. No.: 15/128,010

(22) PCT Filed: Mar. 27, 2015
(86) PCT No.: PCT/CN2015/075298

§ 371 Date: Sep. 21, 2016

(30) Foreign Application Priority Data
Mar. 27, 2014 (CN) PCT/CN2014/074191

(51) Int. Cl. C11B 3/00; C12N 15/52; C12N 9/20

(52) U.S. Cl. CPC C11B 3/003; C12N 9/20; C12Y 301/04003; C12N 15/52

(57) ABSTRACT

The present invention provides polypeptides having phospholipase C activity and polynucleotides encoding the polypeptides. Also provided are nucleic acid constructs, vectors, and host cells comprising the polynucleotides as well as methods of producing the polypeptides. Furthermore, the present invention provides a method of reducing the phospholipid content in an oil composition using the polypeptide having phospholipase C activity as well as other relevant uses of the polypeptide.

### REFERENCE TO A SEQUENCE LISTING

This application contains a Sequence Listing in computer readable form, which is incorporated herein by reference.

### BACKGROUND OF THE INVENTION

Field of the Invention

The present invention relates to polypeptides having phospholipase C activity and polynucleotides encoding the polypeptides. The invention also relates to nucleic acid constructs, vectors, and host cells comprising the polynucleotides as well as methods of producing and using the polypeptides. Further, the present invention relates to a method of reducing the phospholipid content in an oil composition using the polypeptide having phospholipase C activity.

Description of the Related Art

Several types of phospholipases are known which differ in their specificity according to the position of the bond attacked in the phospholipid molecule. Phospholipase A1 (PLA1) removes the 1-position fatty acid to produce free fatty acid and 1-lyso-2-acylphospholipid. Phospholipase A2 (PLA2) removes the 2-position fatty acid to produce free fatty acid and 1-acyl-2-lysophospholipid. The term phospholipase B (PLB) is used for phospholipases having both A1 and A2 activity. Phospholipase C (PLC) removes the phosphate moiety to produce 1,2 diacylglycerol and phosphate ester. Phospholipase D (PLD) produces 1,2-diacylglycero-phosphate and base group (See FIG. 1).

Before consumption vegetable oils are degummed to provide refined storage stable vegetable oils of neutral taste and light color. The degumming process comprises removing the phospholipid components (the gum) from the triglyceride rich oil fraction. The most commonly used processes in the industry are water degumming, chemical/caustic refining and physical refining including acid assisted degumming and/or enzyme assisted degumming. Due to the emulsifying properties of the phospholipid components, the degumming procedure has resulted in a loss of oil; i.e. of triglycerides.

Enzymatic degumming reduces the oils loss due to an efficient hydrolysis of the phospholipids which decrease the emulsifying properties. For a review on enzymatic degumming see Dijkstra 2010 Eur. J. Lipid Sci. Technol. 112, 1178. The use of Phospholipase A and/or phospholipase C in degumming is for example described in Clausen 2001 Eur J Lipid Sci Techno 103 333-340, WO 2003/089620 and WO 2008/094847. Phospholipase A solutions generate lysophospholipid and free fatty acids resulting in oil loss. Phospholipase C on the other hand has the advantage that it produces diglyceride (FIG. 2) which will remain in the oil and therefore will reduce losses. There are four major phospholipids in vegetable oil phosphatidylcholine (PC), phosphatidylethanolamine (PE), phosphatidic acid (PA) and phosphatidyl inositol (PI). Phospholipase C enzymes have different specificity towards these phospholipids. The only known commercially available phospholipase C is Purifine of Verenium/DSM (Dijkstra, 101st AOCS Annual Meeting 10. May 2010) which has specificity towards PC and PE. WO07/059927 describes a thermostable Bacillus PLC for degumming. WO 2012/062817 describes a fungal PLC with specificity towards all four phospholipids.

### BRIEF DESCRIPTION OF THE FIGURES

FIG. 1 illustrates where different phospholipases cleave a phospholipid as well as the four major functional groups on phospholipids.

FIG. 2 illustrates the reaction of a phospholipid with a phospholipase C to form diglyceride and a phosphate ester or phosphoric acid.

### SUMMARY OF THE INVENTION

The present invention provides a polypeptide having phospholipase C activity, selected from the group consisting of: a polypeptide having at least 70% sequence identity to the mature polypeptide of SEQ ID NO: 3;
a) a polypeptide encoded by a polynucleotide that hybridizes under low stringency conditions with (i) the mature polypeptide coding sequence of SEQ ID NO: 1, ii) the cDNA sequence thereof or iii) the full-length complement of i) or ii);
b) a polypeptide encoded by a polynucleotide having at least 70% sequence identity to the mature polypeptide coding sequence of SEQ ID NO: 1 or the cDNA sequence thereof;
c) a variant of the mature polypeptide of SEQ ID NO: 3 comprising a substitution, deletion, and/or insertion at one or more positions; and
d) a fragment of the polypeptide of (a), (b), (c), or (d) that has phospholipase C activity.

Other aspects of the invention include compositions comprising the polypeptide of the invention, polynucleotides encoding the polypeptide of the invention and nucleic acid constructs and expression vectors comprising the polynucleotides, recombinant host cells comprising the polynucleotides, methods of producing the polypeptide and use of the polypeptides or compositions in a process for hydrolysis of phospholipids.

Finally, the invention provides methods for reducing the content of phospholipids in an oil.

### DEFINITIONS

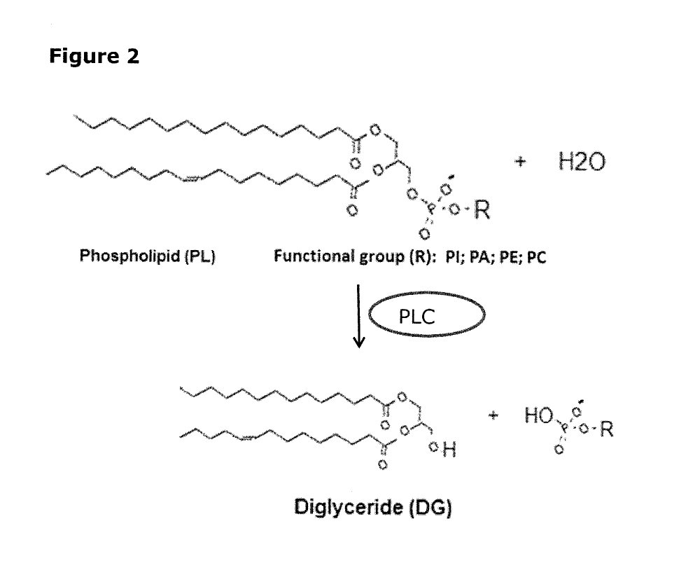

Phospholipase C activity: The term “phospholipase C activity” or “PLC activity” relates to an enzymatic activity that removes the phosphate ester moiety from a phospholipid to produce a 1,2 diacylglycerol (see FIG. 2). Most PLC enzymes belong to the family of hydrolases and phosphodiesterases and are generally classified as EC 3.1.4.3. Phospholipase C activity may be determined according to the procedure described in Example 5 or by one of the assays described in the “Assay for phospholipase activity” section.

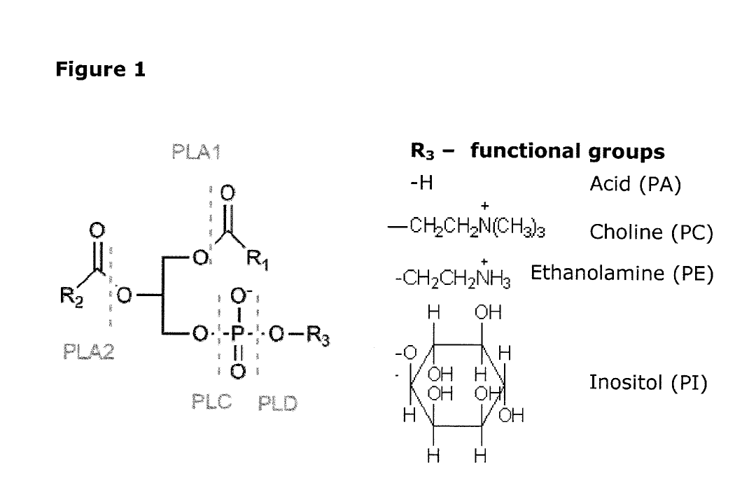

Phospholipase C specificity: The term “phospholipase C specificity” relate to a polypeptide having phospholipase C activity where the activity is specified towards one or more phospholipids, with the four most important once being phosphatidylcholine (PC), phosphatidylethanolamine (PE), phosphatidic acid (PA) and phosphatidyl inositol (PI) (see FIG. 1). Phospholipase C specificity may be determined by ³¹P-NMR as described in Example 5.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Crude oil: The term “crude oil” refers to (also called a non-degummed oil) a pressed or extracted oil or a mixture thereof from, e.g. vegetable sources, including but not limited to acai oil, almond oil, babassu oil, blackcurrent seed oil, borage seed oil, canola oil, cashew oil, castor oil, coconut oil, coriander oil, corn oil, cottonseed oil, crambe oil, flax seed oil, grape seed oil, hazelnut oil, hempseed oil, jatropha oil, jojoba oil, linseed oil, macadamia nut oil, mango kernel oil, meadowfoam oil, mustard oil, neat's foot oil, olive oil, palm oil, palm kernel oil, palm olein, peanut oil, pecan oil, pine nut oil, pistachio oil, poppy seed oil, rapeseed oil, rice bran oil, safflower oil, sasanqua oil, sesame oil, shea butter, soybean oil, sunflower seed oil, tall oil, tsubaki oil walnut oil, varieties of “natural” oils having altered fatty acid compositions via Genetically Modified Organisms (GMO) or traditional “breading” such as high oleic, low linolenic, or low saturated oils (high oleic canola oil, low linolenic soybean oil or high stearic sunflower oils).

Degummed oil: The term “degummed oil” refers to an oil obtained after removal of nonhydratable phospholipids, hydratable phospholipids, and lecithins (known collectively as “gums”) from the oil to produce a degummed oil or fat product that can be used for food production and/or non-food applications, e.g. biodiesel. In certain embodiments, the degummed oil has the phospholipids content of less than 200 ppm phosphorous, such as less than 150 ppm phosphorous, less than 100 ppm phosphorous, less than (or less than about) 50 ppm phosphorous, less than (or less than about) 40 ppm phosphorous, less than (or less than about) 30 ppm phosphorous, less than (or less than about) 20 ppm phosphorous, less than (or less than about) 15 ppm phosphorous, less than (or less than about) 10 ppm phosphorous, less than (or less than about) 7 ppm phosphorous, less than (or less than about) 5 ppm phosphorous, less than (or less than about) 3 ppm phosphorous or less than (or less than about) 1 ppm phosphorous.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has phospholipase C activity. The fragments according to the invention have a size of more than approximately 200 amino acid residues, preferably more than 250 amino acid residues, more preferred more than 300 amino acid residues, more preferred more than 350 amino acid residues, more preferred more than 400 amino acid residues, more preferred more than 450 amino acid residues, more preferred more than 500 amino acid residues, more preferred more than 550 amino acid residues (e.g., amino acids 40 to 590 of SEQ ID NO: 3 or amino acid 37 to 587 of SEQ ID NO: 3), and most preferred more than 560 amino acid residues. In one aspect, a fragment contains at least 570 amino acid residues (e.g., amino acids 37 to 607 or amino acid 62 to 632 of SEQ ID NO: 3), at least 580 amino acid residues (e.g., amino acids 37 to 617 or amino acid 52 to 632 of SEQ ID NO: 3), or at least 585 amino acid residues (e.g., amino acids 37 to 622 or amino acid 47 to 632 of SEQ ID NO: 3).

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 20 to 632 of SEQ ID NO: 3 based on the Signal P version 3 program (Nielsen et al., 1997, Protein Engineering 10: 1-6) which predicts that amino acids 1 to 19 of SEQ ID NO: 3 are a signal peptide. When expressed in Aspergillus as described in Example 1 the N-terminal sequence was identified to be DWVEDLW (See Example 3, corresponding to amino acids 37 to 43 of SEQ ID NO: 3). This indicates the presence of a propeptide from amino acid sequence 20 to 36 which is cleaves of during expression in Aspergillus. In another aspect the mature polypeptide is amino acids 37 to 632 of SEQ ID NO: 3. It is known in the art that a host cell may produce a mixture of two or more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having phospholipase activity. In one aspect, the mature polypeptide coding sequence is nucleotides 58 to 2112 of SEQ ID NO: 1 or the cDNA sequence thereof (SEQ ID NO: 2). SignalP (Nielsen et al., 1997 Protein Engineering 10: 1-6) predicts that nucleotides 1 to 57 of SEQ ID NO: 1 encode a signal peptide. In another aspect, the mature polypeptide coding sequence is nucleotides 109 to 2112 of SEQ ID NO: 1. In another aspect the introns of SEQ ID NO: 1 are predicted by Agene to be nucleotides 169 to 222, 310 to 360, 666 to 727 and 937 to 985 of SEQ ID NO: 1.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Variant: The term “variant” means a polypeptide having phospholipase C activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

### DETAILED DESCRIPTION OF THE INVENTION

The present invention relates to a phospholipase C enzyme obtained from a fungus, preferably a fungus of the genus Nectria, more preferably of the species Nectria mariannaeae. The phospholipase C enzyme of the present invention derived showed specificity toward all four major phospholipids in oils, namely phosphatidic acid (PA), phosphatidylcholine (PC), phosphatidylethanolamine (PE) and phosphatidyl inositol (PI).

The present invention furthermore relates to a method for reducing the content of phospholipids in an oil composition using fungal phospholipase C enzyme of the present invention.

### Polypeptides Having Phospholipase C Activity

An aspect of the present invention relates to a polypeptide having phospholipase C activity, selected from the group consisting of: a) a polypeptide having at least 70% sequence identity to the mature polypeptide of SEQ ID NO: 3; b) a polypeptide encoded by a polynucleotide that hybridizes under low stringency conditions with i) the mature polypeptide coding sequence of SEQ ID NO: 1, ii) the cDNA sequence thereof (SEQ ID NO: 2) or iii) the full-length complement of i) or ii); c) a polypeptide encoded by a polynucleotide having at least 70% sequence identity to the mature polypeptide coding sequence of SEQ ID NO: 1 or the cDNA sequence thereof (SEQ ID NO: 2); d) a variant of the mature polypeptide of SEQ ID NO: 3 comprising a substitution, deletion, and/or insertion at one or more positions; and e) a fragment of the polypeptide of (a), (b), (c), or (d) that has PC and PE specific phospholipase C activity.

In one embodiment, the present invention relates to polypeptides having a sequence identity to the mature polypeptide of SEQ ID NO: 3 of at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have phospholipase C activity. In one aspect, the polypeptides differ by up to 10 amino acids, e.g., 1, 2, 3, 4, 5, 6, 7, 8, 9, or 10, from the mature polypeptide of SEQ ID NO: 3.

In an embodiment, the polypeptide has been isolated. A polypeptide of the present invention preferably comprises, consists of or consists essentially of the amino acid sequence of SEQ ID NO: 3 or an allelic variant thereof; or is a fragment thereof having phospholipase C activity. In another aspect, the polypeptide comprises, consists of or consists essentially of the mature polypeptide of SEQ ID NO: 3. In another aspect, the polypeptide comprises, consists of or consists essentially of amino acids 20 to 632 of SEQ ID NO: 3 or amino acids 37 to 632 SEQ ID NO: 3.

In another embodiment the phospholipase C polypeptide of the present invention is a fragment of more than 550 amino acids and the fragment has phospholipase C activity.

In particular, the polypeptide may have a length of 570-620 amino acid residues, such as a length of 570-610 amino acid residues, 570-605 amino acid residues, 570-600 amino acid residues, 570-598 amino acid residues 570-597 amino acid residues, 570-596 amino acid residues, 580-620 amino acid residues, 580-615 amino acid residues, 580-610 amino acid residues, 580-605 amino acid residues, 580-600 amino acid residues, 580-698 amino acid residues, 580-597 amino acid residues, 580-596 amino acid residues, 590-620 amino acid residues, 590-615 amino acid residues, 590-610 amino acid residues, 590-605 amino acid residues, 590-600 amino acid residues, 590-698 amino acid residues, 590-597 amino acid residues, 590-596 amino acid residues, 595-620 amino acid residues, 595-615 amino acid residues, 595-610 amino acid residues, 595-605 amino acid residues, 595-600 amino acid residues, 595-598 amino acid residues, 595-597 amino acid residues, or a length of 595-596 amino acid residues.

In a preferred embodiment the phospholipase C polypeptide of the invention has activity towards all of the following phospholipids, phosphatidic acid (PA), phosphatidylcholine (PC), phosphatidylethanolamine (PE) and phosphatidyl inositol (PI). Preferably, the polypeptide of the present invention is a PA, PC, PE and PI specific phospholipase C enzyme. The phospholipase C polypeptide of the present invention is capable of reducing the PA, PC, PE, and PI content in an oil. Preferably, the polypeptide of the invention is capable of reducing the PA content in an oil by at least 30% when applied in 10 mg Enzyme Protein/kg oil at the optimal pH of the polypeptide, more preferred at least 35%, 40%, 45%, 50%, 55%, 60%, 65%, 70%, 75%, 80%, 85%, 90%, 95%, 99% or 100%. Preferably, the polypeptide of the invention is capable of reducing the PC content in an oil by at least 30% when applied in 10 mg Enzyme Protein/kg oil at the optimal pH of the polypeptide, more preferred at least 35%, 40%, 45%, 50%, 55%, 60%, 65%, 70%, 75%, 80%, 85%, 90%, 95%, 99% or 100%. Preferably, the polypeptide of the invention is capable of reducing the PI content in an oil by at least 30% when applied in 10 mg Enzyme Protein/kg oil at the optimal pH of the polypeptide, more preferred at least 35%, 40%, 45%, 50%, 55%, 60%, 65%, 70%, 75%, 80%, 85%, 90%, 95%, 99% or 100%. Preferably, the polypeptide of the invention is capable of reducing the PE content in an oil by at least 30% when applied in 10 mg Enzyme Protein/kg oil at the optimal pH of the polypeptide, more preferred at least 35%, 40%, 45%, 50%, 55%, 60%, 65%, 70%, 75%, 80%, 85%, 90%, 95%, 99% or 100%.

In a further embodiment the optimal pH range of polypeptide of the present invention is between 4.0 to 8.5, more preferred from 4.5 to 8.0, even more preferred from 5.0 to 7.5, most preferred from pH 5.5 to 7.0.

The reduction of PA, PC, PE and PI content may in particular be determined by ³¹P-NMR after addition of 100 mg enzyme protein (EP)/kg oil and incubation of the oil and enzyme at 50° C. for 2 hours at pH 5.5.

The ability of the polypeptide to reduce phosphorous content and increase dicylglyceride content may in particular be determined using a crude soy oil which comprises 80-140 ppm phosphorous present as phosphatidic acid (PA), 140-200 ppm phosphorous present as phosphatidyl ethanolamine (PE), 70-110 ppm phosphorous present as phosphatidic acid (PI) and 130-200 ppm phosphorous present as phosphatidyl choline; the phosphorous content being measured by ³¹P-NMR and the diacylglycerol content being measured by HPLC-Evaporative Light Scattering Detection (HPLC-ELSD).

In particular, the polypeptide of the present invention is capable of increasing the amount of diacylglyceride by at least 0.1% w/w when applied in amounts of 8.5 mg EP/kg oil to crude soy bean oil and incubated for 3 hours. Preferably, the oil has been acid/base treated by addition of 85% solution of Ortho Phosphoric acid in amounts corresponding to 0.05% (100% pure Ortho Phosphoric acid) based on oil amount, and base neutralization with 4 M NaOH applied in equivalents of 0.5 to pure Ortho Phosphoric acid.

Preferably, the polypeptide of the present invention is capable of increasing the amount of diacylglyceride by at least 0.3% w/w when applied in amounts of 8.5 mg EP/kg to crude soybean oil and incubated with the oil for 3 hours at 50° C., when the crude oil has been acid/base treated with 0.05% Ortho Phosphoric acid and 1 eqv. NaOH.

The reduction of PA, PC, PE and PI content and/or production of diacylglyceride may in particular be obtained in an oil degumming process comprising the steps of:
i) Optionally treating crude soy bean oil with acid/base by adding an 85% solution of Ortho Phosphoric acid in amounts corresponding to 0.05% (100% pure Ortho Phosphoric acid) based on oil amount, mixing in ultrasonic bath for 5 minutes, followed by incubation in rotator for 15 minutes and base neutralization with 4 M NaOH applied in equivalents (from 0.5 to 0.15) to pure Ortho Phosphoric acid in ultrasonic bath for 5 minutes;
ii) Adding the polypeptide to the oil in a low aqueous system comprising 3% water based on oil amount and subjecting the oil and the polypeptide to ultrasonic treatment for 5 minutes;
iii) Incubating the polypeptide and oil at 50-60° C. with stirring at 20 rpm;
iv) Centrifuging the oil and the polypeptide at 700 g at 85° C. for 15 minutes.

The closest related sequence to the phospholipase C polypeptide of the present invention is UniProt nr C7YU99 with 64.1% identity to the mature sequence of SEQ ID NO: 3 (amino acids 37 to 632). To our knowledge UniProt nr C7YU99 has never been expressed and characterized and its use in degumming or any other application has never been described. The most closely related phospholipase C which has been described in degumming is the Kionochaeta PLC in WO 2012/062817 (indicated as SEQ ID NO: 4 herein) with 59.8% identity to the mature sequence of SEQ ID NO: 3 (amino acids 37 to 632).

In another embodiment, the present invention relates to a polypeptide having phospholipase C activity encoded by a polynucleotide that hybridizes under low stringency conditions, medium stringency conditions, medium-high stringency conditions, high stringency conditions, or very high stringency conditions with (i) the mature polypeptide coding sequence of SEQ ID NO: 1, (ii) the cDNA sequence thereof or (iii) the full-length complement of (i) or (ii) (Sambrook et al., 1989, Molecular Cloning, A Laboratory Manual, 2d edition, Cold Spring Harbor, N.Y.). In an embodiment, the polypeptide has been isolated.

The polynucleotide of SEQ ID NO: 1 or the cDNA thereof or a subsequence thereof, as well as the polypeptide of SEQ ID NO: 3 or a fragment thereof, may be used to design nucleic acid probes to identify and clone DNA encoding polypeptides having phospholipase C activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having PC and PE-specific phospholipase C activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) SEQ ID NO: 1; (ii) the mature polypeptide coding sequence of SEQ ID NO: 1; (iii) the cDNA sequence thereof (iv) the full-length complement thereof; or (v) a subsequence thereof; under low to very high stringency conditions. Molecules to which the nucleic acid probe hybridizes under these conditions can be detected using, for example, X-ray film or any other detection means known in the art.

In another embodiment, the present invention relates to a polypeptide having phospholipase C activity encoded by a polynucleotide having a sequence identity to the mature polypeptide coding sequence of SEQ ID NO: 1 or the cDNA thereof of at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 3 comprising a substitution, deletion, and/or insertion at one or more (e.g., several) positions. In an embodiment, the number of amino acid substitutions, deletions and/or insertions introduced into the mature polypeptide of SEQ ID NO: 3 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

In relation to the polypeptides of the present invention examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Other possible approaches to generating variants having similar or substantially similar physic-chemical or functional properties as mature polypeptide of SEQ ID NO: 3 would include introducing changes in the amino acid sequence within regions showing medium to high variability, identified by aligning the amino acid sequence of SEQ ID NO: 3 with closest related sequences, including SWISSPROT:R8BJZ4, SWISSPROT:N1RI50, SWISSPROT:K3UWT8, SWISSPROT:I1RS88, SWISSPROT:E9ELM8 and the fungal kionochaeta PLC of SEQ ID NO: 4. By such alignment the following regions having medium or high variability may be identified in mature polypeptide of SEQ ID NO: 3 (using the amino acid numbering of SEQ ID NO: 3):

| | |
|---|---|
| Amino acids 37-89 | High variability |
| Amino acids 90-104 | Medium variability |
| Amino acids 105-156 | High variability |
| Amino acids 168-179 | Medium variability |
| Amino acids 188-194 | High variability |
| Amino acids 205-227 | Medium variability |
| Amino acids 250-267 | High variability |
| Amino acids 280-303 | Medium variability |
| Amino acids 304-321 | High variability |
| Amino acids 327-330 | Medium variability |
| Amino acids 342-357 | Medium variability |
| Amino acids 360-371 | Medium variability |
| Amino acids 422-428 | Medium variability |
| Amino acids 430-442 | High variability |
| Amino acids 482-486 | High variability |
| Amino acids 514-515 | High variability |
| Amino acids 528-544 | High variability |
| Amino acids 549-569 | High variability |
| Amino acids 575-632 | High variability |

Hence, in some embodiments of the invention present invention relates to a polypeptide having a sequence identity to the mature polypeptide of SEQ ID NO: 3 of at least 70%, at least 75%, at least 80%, at least 85%, at least 90%, at least 91%, at least 92%, at least 93%, at least 94%, at least 95%, at least 96%, at least 97%, at least 98%, at least 99%, wherein the polypeptide has phospholipase C activity and wherein one or more amino acids residues, such as 1, 2, 3, 4, 5, 6, 7, 8, 9, or 10 amino acid residues, have been substituted, deleted or added in the region defined by amino acids 37-89; one or more amino acids residues, such as 1, 2, 3, 4, 5, 6, 7, 8, 9, or 10 amino acid residues, have been substituted, deleted or added in the region defined by amino acids 90-104; one or more amino acids residues, such as 1, 2, 3, 4, 5, 6, 7, 8, 9, or 10 amino acid residues, have been substituted, deleted or added in the region defined by amino acids 105-156, one or more amino acids residues, such as 1, 2, 3, 4, 5, 6, 7, 8, 9, or 10 amino acid residues, have been substituted, deleted or added in the region defined by amino acids 168-179; one or more amino acids residues, such as 1, 2, 3, 4, 5, 6, 7, 8, 9, or 10 amino acid residues, have been substituted, deleted or added in the region defined by amino acids 188-194; one or more amino acids residues, such as 1, 2, 3, 4, 5, 6, 7, 8, 9, or 10 amino acid residues, have been substituted, deleted or added in the region defined by amino acids 205-227; one or more amino acids residues, such as 1, 2, 3, 4, 5, 6, 7, 8, 9, or 10 amino acid residues, have been substituted, deleted or added in the region defined by amino acids 250-267; one or more amino acids residues, such as 1, 2, 3, 4, 5, 6, 7, 8, 9, or 10 amino acid residues, have been substituted, deleted or added in the region defined by amino acids 280-303, one or more amino acids residues, such as 1, 2, 3, 4, 5, 6, 7, 8, 9, or 10 amino acid residues, have been substituted, deleted or added in the region defined by amino acids 304-321, one or more amino acids residues, such as 1, 2, 3, 4, 5, 6, 7, 8, 9, or 10 amino acid residues, have been substituted, deleted or added in the region defined by amino acids amino acids 327-330, one or more amino acids residues, such as 1, 2, 3, 4, 5, 6, 7, 8, 9, or 10 amino acid residues, have been substituted, deleted or added in the region defined by amino acids 342-357; one or more amino acids residues, such as 1, 2, 3, 4, 5, 6, 7, 8, 9, or 10 amino acid residues, have been substituted, deleted or added in the region defined by amino acids 360-371; one or more amino acids residues, such as 1, 2, 3, 4, 5, 6, 7, 8, 9, or 10 amino acid residues, have been substituted, deleted or added in the region defined by amino acids 422-428; one or more amino acids residues, such as 1, 2, 3, 4, 5, 6, 7, 8, 9, or 10 amino acid residues, have been substituted, deleted or added in the region defined by amino acids 430-442; one or more amino acids residues, such as 1, 2, 3, 4, 5, 6, 7, 8, 9, or 10 amino acid residues, have been substituted, deleted or added in the region defined by amino acids 482-486; one or more amino acids residues, such as 1, 2, 3, 4, 5, 6, 7, 8, 9, or 10 amino acid residues, have been substituted, deleted or added in the region defined by amino acids 514-515; one or more amino acids residues, such as 1, 2, 3, 4, 5, 6, 7, 8, 9, or 10 amino acid residues, have been substituted, deleted or added in the region defined by amino acids 528-544; one or more amino acids residues, such as 1, 2, 3, 4, 5, 6, 7, 8, 9, or 10 amino acid residues, have been substituted, deleted or added in the region defined by amino acids 549-569 and/or one or more amino acids residues, such as 1, 2, 3, 4, 5, 6, 7, 8, 9, or 10 amino acid residues, have been substituted, deleted or added in the region defined by amino acids 575-632; using the amino acids numbering of SEQ ID NO: 3.

Preferably, the polypeptide has specificity towards all four phospholipid species: phosphatidic acid (PA), phosphatidylcholine (PC), phosphatidylethanolamine (PE) and phosphatidyl inositol (PI) as described above. In equally preferred embodiments the polypeptide performs in degumming; i.e. is capable of increasing the amount of diacylglyceride, when applied to crude soybean oil as described above.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for the desired phospholipase C activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Essential amino acids in the polypeptide encoded by SEQ ID NO: 3 are predicted to be located at positions D165, H167, D239, N277, H385, H419 and H421. These amino acids are believed to be involved in coordinating the three Zn ions needed for the catalytic activity based on the homology model of the sequence SEQ ID NO: 3. In a preferred embodiment a polypeptide of the invention maintain the amino acids corresponding to position 165, 167, 239, 277, 385, 419 and 421 when aligned to SEQ ID NO: 3.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie at al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Phospholipase C Activity

A polypeptide having phospholipase C activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. For example, the polypeptide may be from a Ascomycota. Preferably the polypeptide is from the genus of Nectria.

In one aspect, the polypeptide is a Nectria mariannaeae or Nectria haematococca polypeptide.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Nectria, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1 or the cDNA of SEQ ID NO: 1, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in an expression host. Preferably the expression is done in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

The control sequence may also be an mRNA stabilizer region downstream of a promoter and upstream of the coding sequence of a gene which increases expression of the gene.

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. Preferably the polynucleotide is heterologous, meaning that it does not exist naturally in the host cell. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote. In a preferred embodiment the host cell is a recombinant host cell which does not exist in nature. The host cell may also be a eukaryote, such as a mammalian, insect, plant, or fungal cell.

In a preferred embodiment the host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK)

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell. In a preferred embodiment the hos cell is an Aspergillus niger or Aspergillus oryzae cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Nectria cell. In another aspect, the cell is a Nectria mariannaeae cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide with phospholipase C activity may be detected using methods known in the art, see the “Assay for phospholipase activity” section below. These detection methods include, but are not limited to, use of specific antibodies, formation of an enzyme product, or disappearance of an enzyme substrate e.g. P-NMR assay described in example 5 or liquid chromatography coupled to triple quadrupole mass spectrometer (LC/MS/MS) as described in Example 6 or p-Nitrophenylphosphorylcholine assays or plate assays as described in the “Assay for phospholipase activity” section.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Assays for Phospholipase Activity

The invention provides isolated, synthetic or recombinant polypeptides (e.g., enzymes, antibodies) having a phospholipase activity, or any combination of phospholipase activities, and nucleic acids encoding them. Any of the many phospholipase activity assays known in the art can be used to determine if a polypeptide has a phospholipase activity and is within the scope of the invention. Routine protocols for determining phospholipase A, B, D and C, are well known in the art.

Exemplary activity assays include turbidity assays, methylumbelliferyl phosphocholine (fluorescent) assays, Amplex red (fluorescent) phospholipase assays, thin layer chromatography assays (TLC), cytolytic assays and p-nitrophenylphosphorylcholineassays. Using these assays polypeptides, peptides or antibodies can be quickly screened for a phospholipase activity.

Plate assays with a substrate containing agar can be used to determine phospholipase activity Plate assay. Useful substrates are lecithin or specific phospholipids. The assay can be conducted as follows. Plates are casted by mixing of 5 ml 2% Agarose (Litex HSA 1000) prepared by mixing and cooking in buffers (100 mM HEPES and 100 mM Citrate with pH adjusted from pH 3.0 to pH 7.0) for 5 minutes followed by cooling to approximately 60° C. and 5 ml substrate (L-alfa Phosohatidylcholine, 95% from Soy (Avanti 441601) or L-α-phosphatidylinositol from Soy (Avanti 840044P) for PI-specificity or L-α-phosphatidylethanolamine from Soy (Avanti 840024P) or lecithin) dispersed in water (MilliQ) at 60° C. for 1 minute with Ultra Turrax for PC-specificity) gently mixed into petri dishes with diameter of 7 cm and cooled to room temperature before holes with a diameter of approximately 3 mm were punched by vacuum. Ten microliters of purified enzyme diluted to 0.4 mg/ml is added into each well before plates were sealed by parafilm and placed in an incubator at 55° C. for 48 hours. Plates were taken out for photography at regular intervals.

Turbidity assays to determine phospholipase activity are described, e.g., in Kauffmann (2001) “Conversion of Bacillus thermocatenulatus lipase into an efficient phospholipase with increased activity towards long-chain fatty acyl substrates by directed evolution and rational design,” Protein Engineering 14:919-928; Ibrahim (1995) “Evidence implicating phospholipase as a virulence factor of Candida albicans,” Infect. Immun. 63:1993-1998.

Methylumbelliferyl (fluorescent) phosphocholine assays to determine phospholipase activity are described, e.g., in Goode (1997) “Evidence for cell surface internal phospholipase activity in ascidian eggs,” Develop. Growth Differ. 39:655-660; Diaz (1999) “Direct fluorescence-based lipase activity assay,” BioTechniques 27:696-700.

Amplex Red (fluorescent) Phospholipase Assays to determine phospholipase activity are available as kits, e.g., the detection of phosphatidylcholine-specific phospholipase using an Amplex Red phosphatidylcholine-specific phospholipase assay kit from Molecular Probes Inc. (Eugene, Oreg.), according to manufacturer's instructions.

Fluorescence is measured in a fluorescence microplate reader using excitation at 560±10 nm and fluorescence detection at 590±10 nm. The assay is sensitive at very low enzyme concentrations.

Thin layer chromatography assays (TLC) to determine phospholipase activity are described, e.g., in Reynolds (1991) Methods in Enzymol. 197:3-13; Taguchi (1975) “Phospholipase from Clostridium novyi type A.I,” Biochim. Biophys. Acta 409:75-85. Thin layer chromatography (TLC) is a widely used technique for detection of phospholipase activity. Various modifications of this method have been used to extract the phospholipids from the aqueous assay mixtures. In some PLC assays the hydrolysis is stopped by addition of chloroform/methanol (2:1) to the reaction mixture. The unreacted starting material and the diacylglycerol are extracted into the organic phase and may be fractionated by TLC, while the head group product remains in the aqueous phase. For more precise measurement of the phospholipid digestion, radio labeled substrates can be used (see, e.g., Reynolds (1991) Methods in Enzymol. 197:3-13). The ratios of products and reactants can be used to calculate the actual number of moles of substrate hydrolyzed per unit time. If all the components are extracted equally, any losses in the extraction will affect all components equally. Separation of phospholipid digestion products can be achieved by silica gel TLC with chloroform/methanol/water (65:25:4) used as a solvent system (see, e.g., Taguchi (1975) Biochim. Biophys. Acta 409:75-85).

p-Nitrophenylphosphorylcholine assays to determine phospholipase activity are described, e.g., in Korbsrisate (1999) J. Clin. Microbiol. 37:3742-3745; Berka (1981) Infect. Immun. 34:1071-1074. This assay is based on enzymatic hydrolysis of the substrate analog p-nitrophenylphosphorylcholine to liberate a yellow chromogenic compound p-nitrophenol, detectable at 405 nm. This substrate is convenient for high throughput screening. Similar assays using substrates towards the other phospholipid groups can also be applied e.g. using p-nitrophenylphosphorylinositol or p-nitrophenylphosphorylethanolamine.

A cytolytic assay can detect phospholipases with cytolytic activity based on lysis of erythrocytes. Toxic phospholipases can interact with eukaryotic cell membranes and hydrolyze phosphatidylcholine and sphingomyelin, leading to cell lysis. See, e.g., Titball (1993) Microbiol. Rev. 57:347-366.

Further assays like ³¹P-NMR and Liquid Chromatography coupled to triple quadrupole mass spectrometer (LC/MS/MS) are described in the example section of this application.

### Compositions

The present invention also relates to compositions comprising a phospholipase polypeptide of the present invention, preferably with an additional component. Preferably the composition comprises at least 1 mg of the phospholipase of the present invention pr. ml solution, more preferably at least 5 mg/ml, even more preferred at least 10 mg/ml and most preferred at least 15 mg/ml.

The phospholipase of the present invention may be formulated with components selected from the group consisting of buffer agents, inorganic salts, solvents, inert solids and mixtures thereof. Appropriate buffer systems, e.g., are made from aqueous solutions of salts or organic acids, amino acids, phosphate, amines or ammonia in concentrations between 0.01 M and 1 M at pH 2 to 10. Preferably, alkali metal salts of citric acid, acetic acid, glycine and/or hydrochlorides of tris(hydroxymethyl)amine and ammonia at 0.1 M to 0.2 M at pH 4 to 8 are used. Preferably, the phospholipase is dissolved in an aqueous buffer solution such as glycine buffer, citric acid buffer, etc. Citrate containing buffers have been found to be very suitable, in particular sodium citrate buffers, preferably at neutral pH.

The compositions of the invention may comprise a phospholipase of the invention immobilized unto a solid support. Solid supports useful in this invention include gels. Some examples of gels include Sepharose, gelatin, glutaraldehyde, chitosan-treated glutaraldehyde, albumin-glutaraldehyde, chitosan-Xanthan, toyopearl gel (polymer gel), alginate, alginate-polylysine, carrageenan, agarose, glyoxyl agarose, magnetic agarose, dextranagarose, poly(Carbamoyl Sulfonate) hydrogel, BSA-PEG hydrogel, phosphorylated polyvinyl alcohol (PVA), monoaminoethyl-N-aminoethyl (MANA), amino, or any combination thereof. Another solid support useful in the present invention are resins or polymers. Some examples of resins or polymers include cellulose, acrylamide, nylon, rayon, polyester, anion-exchange resin, AMBERLITE™ XAD-7, AMBERLITE™ XAD-8, AMBERLITE™ IRA-94, AMBERLITE™ IRC-50, polyvinyl, polyacrylic, polymethacrylate, or any combination thereof. Another type of solid support useful in the present invention is ceramic. Some examples include non-porous ceramic, porous ceramic, Si02, Ah03. Another type of solid support useful in the present invention is glass. Some examples include non-porous glass, porous glass, aminopropyl glass or any combination thereof. Another type of solid support that can be used is a microelectrode. An example is a polyethyleneimine-coated magnetite. Graphitic particles can be used as a solid support. Other exemplary solid supports used to practice the invention comprise diatomaceous earth products and silicates. Some examples include CELITE® KENITE®, DIACTIV®, PRIMISIL®′ DIAFIL® diatomites and MICRO-CEL®′ CALFLO®, SILASORB™, and CELKA TE® synthetic calcium and magnesium silicates.

Some examples of methods for immobilizing enzymes include, e.g., electrostatic droplet generation, electrochemical means, via adsorption, via covalent binding, via cross-linking, via a chemical reaction or process, via encapsulation, via entrapment, via calcium alginate, or via poly (2-hydroxyethyl methacrylate). Like methods are described in Methods in Enzymology, Immobilized Enzymes and Cells, Part C. 1987. Academic Press. Edited by S. P. Colowick and N. O. Kaplan. Volume 136; and Immobilization of Enzymes and Cells. 1997. Humana Press. Edited by G. F. Bickerstaff. Series: Methods in Biotechnology, Edited by J. M. Walker.

The composition may comprise a polypeptide of the present invention as the major enzymatic component, e.g., a mono-component composition. In a further embodiment the composition may comprise multiple enzymatic activities, such as one or more (e.g., several) enzymes selected from the group consisting of hydrolase, isomerase, ligase, lyase, oxidoreductase, or transferase, e.g., an alpha-galactosidase, alpha-glucosidase, aminopeptidase, amylase, beta-galactosidase, beta-glucosidase, beta-xylosidase, carbohydrase, carboxypeptidase, catalase, cellobiohydrolase, cellulase, chitinase, cutinase, cyclodextrin glycosyltransferase, deoxyribonuclease, endoglucanase, esterase, glucoamylase, invertase, laccase, lipase, mannosidase, mutanase, oxidase, pectinolytic enzyme, peroxidase, phytase, polyphenoloxidase, proteolytic enzyme, ribonuclease, transglutaminase, or xylanase. Preferably the further enzyme may also be a polypeptide having phospholipase A1, A2, B and/or D activity.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The phospholipases or compositions of the invention may be applied in a process for removing phospholipids from an oil, e.g. a vegetable oil, animal oil or fat, tallow, or grease.

Applications in which the phospholipase of the invention can be used comprise i) degumming of oil, e.g. vegetable oil, or an edible vegetable oil, or in a process comprising hydrolysis of phospholipids in the gum fraction from water degumming to release entrapped triglyceride oil, ii) in a process comprising hydrolysis of phospholipids to obtain improved phospholipid emulsifiers, in particular wherein said phospholipid is lecithin, iii) in a process for improving the filterability of an aqueous solution or slurry of carbohydrate origin which contains phospholipid, iv) in a process for the extraction of oil, v) in a process for the production of an animal feed product, vi) in a process for the production of a biofuel, e.g. a biodiesel, vii) in a process for the production of a detergent product, and/or viii) in a process for making a baked product, comprising adding the phospholipase to a dough, and baking the dough to make the baked product.

The phospholipases of the invention may be applied in a process comprising treatment of a phospholipid or lysophospholipid with the phospholipases or compositions of the invention. The phospholipases or compositions react with the phospholipids or lysophospholipid to form monoglyceride or diglyceride and a phosphate ester or phosphoric acid.

### Degumming:

The phospholipases of the invention and combinations thereof may be used for degumming oil, e.g. animal oil or fat, tallow, grease or a vegetable oil, i.e., in a process to reduce the phospholipid content in the oil. The degumming process is applicable to the purification of any edible oil which contains phospholipid, e.g., vegetable oil such as soybean oil, rape seed oil, or sunflower oil or any other oil mentioned under the definition of crude oils.

The phospholipase of the present invention cleaves phospholipids (phosphatidic acid (PA), phosphatidylcholine (PC), phosphatidylethanolamine (PE) and phosphatidyl inositol (PI)) just before the phosphate group into diglyceride and phosphoric acid (from PA) or phosphate ester (from PC, PE or PI). The diglyceride stays in the oil phase (improving oil yield) and the phosphorous-containing moieties separates into the aqueous phase where they are removed as components of the heavy phase during centrifugation. The gum phase (heavy phase) may be treated further with a phospholipase or composition of the present invention to increase hydrolysis of phospholipids in the gum fraction from water degumming to release entrapped triglyceride oil. This is particular useful when de degumming process has not already applied phospholipases. The phospholipase of the invention can be incorporated into either water degumming or a chemical or physical oil refining process, with preferably less than 10%, 9%, 8%, 7%, 6% or 5% water, even more preferably less than 4%, 3% or 2% water, preferably at 50° C. or above, even more preferably at 60° C. or above. In a preferred embodiment the phospholipase of the invention is incorporated into a water degumming process, caustic refining process or acid degumming process.

In another preferred embodiment the phospholipases of the invention are incorporated into a physical refining process applying citric acid or phosphoric acid and sodium hydroxide to facilitate hydratability of insoluble phospholipids and ensure an environment suitable for the enzyme with preferably less than 0.15% citric acid or phosphoric acid even more preferably less than 0.1%, 0.09%, 0.08%, 0.07%, 0.06% or 0.05%; and less than 4%, 3% or 2% water, preferably at 50° C. or above, even more preferably at 60° C. or above.

In other embodiments the degumming process is a caustic refining process or acid degumming process.

An aspect of the present invention is a method for reducing the content of phospholipids in an oil composition, the method comprising a) contacting said oil with a polypeptide of the present invention or with a composition of the present invention, under conditions sufficient for the enzyme to react with the phospholipids to create diglyceride and phosphate ester or phosphoric acid, and; b) separating the phosphate ester or phosphoric acid from the oil composition. Preferably, the oil composition provided for treatment with the phospholipase of the present invention or a composition thereof contains a quantity of phospholipids.

Phospholipids are commonly measured in oil as “phosphorous content” in parts per million. Table 1 sets forth the typical amounts of phospholipids present in the major oilseed crops, and the distribution of the various functional groups as a percentage of the phospholipids present in the oil.

TABLE 1
Typical levels and phospholipid distributions for common oilseeds

| | Soy Oil | Canola Oil | Sunflower Oil |
|---|---|---|---|
| Phosphorous (ppm) | 400-1500 | 200-900 | 300-700 |
| PC % | 12-46 | 25-40 | 29-52 |
| PE % | 8-34 | 15-25 | 17-26 |
| PA % | 2-21 | 10-20 | 15-30 |
| PI % | 2-15 | 2-25 | 11-22 |

The enzyme, compositions and processes of the invention can be used to achieve a more complete degumming of high phosphorous oils, e.g. an oil with more than 200 ppm of phosphorous, preferably more than 300 ppm, 400 ppm, 500 ppm, 600 ppm, 700 ppm, 800 ppm, 900 ppm, even more preferred the oil contains more than 1000 ppm phosphorous.

Preferably, the oil for treatment in a method of the present invention comprises phosphatidic acid (PA), phosphatidylcholine (PC), phosphatidylethanolamine (PE) and phosphatidyl inositol (PI). Preferably the oil contains more than 50 ppm phosphorous originating from phosphatidyl inositol (PI), more preferably it contains more than 75 ppm, 100 ppm, 125 ppm PI, even more preferably it contains more than 150 ppm, most preferably it contains more than 175 ppm phosphorous originating from PI. Preferably the oil contains more than 100 ppm phosphorous originating from phosphatidylcholine (PC), more preferably it contains more than 150 ppm, 200 ppm, 250 ppm PC, even more preferably it contains more than 300 ppm, most preferably it contains more than 400 ppm phosphorous originating from PC. Preferably the oil contains more than 75 ppm phosphorus originating from phosphatidylethanolamine (PE), more preferably it contains more than 100 ppm, 125 ppm, 150 ppm PE, even more preferably it contains more than 200 ppm, most preferably it contains more than 300 ppm phosphorous originating from PE. Preferably the oil contains more than 10 ppm phosphorus originating from phosphatidic acid (PA), more preferably it contains more than 20 ppm, 30 ppm, 50 ppm, 75 ppm, 100 ppm, 125 ppm, 150 ppm PE, even more preferably it contains more than 200 ppm, most preferably it contains more than 300 ppm phosphorous originating from PA.

In a preferred embodiment the oil is an edible oil. More preferred the edible oil is selected from rice bran, rapeseeds, palm, peanuts and other nuts, soybean, corn, canola, and sunflower oils. The phospholipases of the invention can be used in any “degumming” procedure, including water degumming, ALCON oil degumming (e.g., for soybeans), safinco degumming, “super degumming,” UF degumming, TOP degumming, uni-degumming, dry degumming and ENZYMAX™ degumming. See, for example, WO 2007/103005, US 2008/0182322, U.S. Pat. No. 6,355,693, U.S. Pat. No. 6,162,623, U.S. Pat. No. 6,103,505, U.S. Pat. No. 6,001,640, U.S. Pat. No. 5,558,781 and U.S. Pat. No. 5,264,367 for description of degumming processes where phospholipases of the present invention can be applied. Various “degumming” procedures incorporated by the methods of the invention are described in Bockisch, M. (1998) In Fats and Oils Handbook, The extraction of Vegetable Oils (Chapter 5), 345-5 445, AOCS Press, Champaign, Ill. The phospholipases of the invention can be used in the industrial application of enzymatic degumming of triglyceride oils as described, e.g., in EP 513 709. In a further embodiment the oil is selected from crude oil, water degummed oil, caustic refined oil and acid degummed oil. The water-degumming of a crude oil or fat may be achieved by thoroughly mixing hot water and warm oil or fat having a temperature of between 50° C. to 90° C. for 30 to 60 minutes. This process serves to partially remove the hydratable phospholipids. Also, an acid treatment may be performed before the enzymatic degumming, where the acid used is selected from the group consisting of phosphoric acid, acetic acid, citric acid, tartaric acid, succinic acid, and mixtures thereof, in particular a treatment using citric acid or phosphoric acid are preferred. The acid treatment is preferably followed by a neutralization step to adjust the pH between about 4.0 to 7.0, more preferably from 4.5 to 6.5, preferably using NaOH or KOH. The acid treatment serves to chelate metals bound to the phospholipids hereby making a more hydratable form. Preferably, the phospholipases as described herein is added after water degumming or acid treatment of the oil. It is also possible to perform the degumming step using the phospholipases as described herein on a crude oil or fat, i.e. an oil or fat not previously water degummed or acid treated.

In one aspect, the invention provides methods for enzymatic degumming under conditions of low water, e.g., in the range of between about 0.1% to 20% water or 0.5% to 10% water. In one aspect, this results in the improved separation of a heavy phase from the oil phase during centrifugation. The improved separation of these phases can result in more efficient removal of phospholipids from the oil, including both hydratable and nonhydratable phospholipids. In one aspect, this can produce a gum fraction that contains less entrained neutral oil (triglycerides), thereby improving the overall yield of oil during the degumming process. In one aspect, phospholipase of the invention is used to treat oils to reduce gum mass and increase neutral oil gain through reduced oil entrapment. In one aspect, phospholipases of the invention e.g., a polypeptide having PLC activity, are used for diacylglycerol (DAG) production and to contribute to the oil phase.

The phospholipase treatment can be conducted by dispersing an aqueous solution of the phospholipase, preferably as droplets with an average diameter below 10 microM. The amount of water is preferably 0.5-5% by weight in relation to the oil. An emulsifier may optionally be added. Mechanical agitation may be applied to maintain the emulsion. Agitation may be done with a high shear mixer with a tip speed above 1400 cm/s.

In certain embodiments, a suitable oil degumming method comprises a) mixing an aqueous acid with an oil to obtain an acidic mixture having pH of about 1 to 4, b) mixing a base with the acidic mixture to obtain a reacted mixture having pH of about 6-9, and c) degumming the reacted mixture with an enzyme of the present invention to obtain a degummed oil. In certain embodiments, mixing in steps a) and/or b) creates an emulsion that comprises an aqueous phase in average droplet size between about 15 microM to about 45 microM. In certain embodiments, mixing in steps a) and/or b) creates an emulsion that comprises at least about 60% of an aqueous phase by volume in droplet size between about 15 microM to about 45 microM in size, wherein percentage of the aqueous phase is based on the total volume of the aqueous phase. Any acid deemed suitable by one of skill in the art can be used in the methods provided herein. In certain embodiments, the acid is selected from the group consisting of phosphoric acid, acetic acid, citric acid, tartaric acid, succinic acid, and a mixture thereof. Any acid deemed suitable by one of skill in the art can be used in the methods provided herein. In certain embodiments, the base is selected from the group consisting of sodium hydroxide, potassium hydroxide, sodium silicate, sodium carbonate, calcium carbonate, and a combination thereof.

In a preferred embodiment the phospholipase treatment can be conducted at a pH in the range of about 4.0 to 7.0, more preferably from 4.5 to 6.5. The pH is measured in the emulsion or in the interphase between the between oil and aqueous solution. A suitable temperature is generally 30-80° C. In a preferred embodiment the temperature of the oil is between 50 and 70° C., more preferred between 55 and 65° C. and most preferred between 50 and 60° C. In other preferred embodiments the temperature of the oil is between 50 and 60° C., more preferred between 55 and 60° C. and most preferred between 57 and 60° C.

The reaction time will typically be 1-12 hours (e.g., 1-6 hours, or 1-3 hours, most preferred the reaction time is between 1.5 and 4 hours, even more preferred between 1.5 and 2 hours). A suitable enzyme dosage will usually be 0.1-10 mg per liter (e.g., 0.5-5 mg per liter).

In still further embodiments, the oil is contacted with 0.5-200 mg enzyme protein (EP)/Kg oil of said phospholipase; such as with 0.5-100 mg enzyme protein (EP)/Kg oil of said phospholipase, with 0.5-25 mg enzyme protein (EP)/Kg oil of said phospholipase, with 0.5-15 mg enzyme protein (EP)/Kg oil of said phospholipase. with 0.5-10 mg enzyme protein (EP)/Kg oil of said phospholipase, with 0.5-5 mg enzyme protein (EP)/Kg oil of said phospholipase, with 1-200 mg enzyme protein (EP)/Kg oil of said phospholipase, with 1-100 mg enzyme protein (EP)/Kg oil of said phospholipase, with 1-25 mg enzyme protein (EP)/Kg oil of said phospholipase, with 1-15 mg enzyme protein (EP)/Kg oil of said phospholipase, with 1-10 mg enzyme protein (EP)/Kg oil of said phospholipase, with 1-5 mg enzyme protein (EP)/Kg oil of said phospholipase, with 2-200 mg enzyme protein (EP)/Kg oil of said phospholipase, with 2-100 mg enzyme protein (EP)/Kg oil of said phospholipase, with 2-50 mg enzyme protein (EP)/Kg oil of said phospholipase, with 2-25 mg enzyme protein (EP)/Kg oil of said phospholipase, with 2-15 mg enzyme protein (EP)/Kg oil of said phospholipase, with 2-10 mg enzyme protein (EP)/Kg oil of said phospholipase, with 2-7 mg enzyme protein (EP)/Kg oil of said phospholipase, or with 2-5 mg enzyme protein (EP)/Kg oil of said C.

The phospholipase treatment may be conducted batch wise, e.g., in a tank with stirring, or it may be continuous, e.g., a series of stirred tank reactors. The phospholipase treatment may be followed by separation of an aqueous phase and an oil phase. The separation may be performed by conventional means, e.g., centrifugation. When a liquid lipase is used the aqueous phase will contain phospholipase, and the enzyme may be re-used to improve the process economy.

In a preferred embodiment of the present invention the treatment reduces the total phosphorous content of the oil to below 200 ppm, preferably below 100 ppm, below 50 ppm, below 40 ppm, 30 ppm, 20 ppm, 15 ppm, more preferably below 10 ppm, below 9 ppm, below 8 ppm, below 7 ppm, below 6 ppm, most preferably below 5 ppm.

In addition to the phospholipases of the present invention a further enzyme may be applied in the degumming process outlined above. In a preferred embodiment the further enzyme is a polypeptide having phospholipase A1, A2 and/or B activity. A suitable polypeptide having phospholipase A1 activity may be LECITASE ULTRA available from Novozymes A/S.

### Phospholipid Emulsifiers:

The phospholipase of the invention may be used for partial hydrolysis of phospholipids, preferably lecithin, to obtain improved phospholipid emulsifiers. This application is further described in Ullmann's Encyclopedia of Industrial Chemistry (Publisher: VCH Weinheim (1996)), JP patent 2794574, and JP-B 6-087751.

### Filtration:

The phospholipase of the invention can be used to improve the filterability of an aqueous solution or slurry of carbohydrate origin by treating it with the phospholipase. This is particularly applicable to a solution of slurry containing a starch hydrolyzate, especially a wheat starch hydrolyzate, since this tends to be difficult to filter and to give cloudy filtrates. The treatment can be done in analogy with EP 219,269 (CPC International).

### Animal Feed:

The phospholipase of the invention may be used in a process for the production of an animal feed which comprises mixing the phospholipase with feed substances comprising at least one phospholipid. This can be done in analogy with EP 743 017.

### Biodiesel:

The phospholipase of the present invention may be used in combination with one or more lipolytic enzymes to convert fats and oils to fatty acid alkyl esters while achieving degumming in the same process. Such a process is for example described in U.S. Pat. No. 8,012,724.

### Detergent:

The phospholipase of the invention may be added to and thus be used as a component of a detergent composition.

The detergent composition may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

### Baking:

The phospholipase of the invention may be used for production of dough and baked products from dough, as well as for production of baking compositions and baking additives.

The dough generally comprises wheat meal or wheat flour and/or other types of meal, flour or starch such as corn flour, corn starch, rye meal, rye flour, oat flour, oat meal, soy flour, sorghum meal, sorghum flour, potato meal, potato flour or potato starch.

The dough may be fresh, frozen or par-baked.

The dough is normally leavened dough or dough to be subjected to leavening. The dough may be leavened in various ways, such as by adding chemical leavening agents, e.g., sodium bicarbonate or by adding a leaven (fermenting dough), but it is preferred to leaven the dough by adding a suitable yeast culture, such as a culture of Saccharomyces cerevisiae (baker's yeast), e.g. a commercially available strain of S. cerevisiae.

The dough may also comprise other conventional dough ingredients, e.g.: proteins, such as milk powder, gluten, and soy; eggs (either whole eggs, egg yolks or egg whites); an oxidant such as ascorbic acid, potassium bromate, potassium iodate, azodicarbonamide (ADA) or ammonium persulfate; an amino acid such as L-cysteine; a sugar; a salt such as sodium chloride, calcium acetate, sodium sulfate or calcium sulfate.

The dough may comprise fat (triglyceride) such as granulated fat or shortening, but the invention is particularly applicable to a dough where less than 1% by weight of fat is added, and particularly to a dough which is made without addition of fat.

The dough may further comprise an emulsifier such as mono- or diglycerides, diacetyl tartaric acid esters of mono- or diglycerides, sugar esters of fatty acids, polyglycerol esters of fatty acids, lactic acid esters of monoglycerides, acetic acid esters of monoglycerides, polyoxyethylene stearates, or lysolecithin.

The dough may be used for any kind of baked product prepared from dough, either of a soft or a crisp character, either of a white, light or dark type. Examples are bread (in particular white, whole-meal or rye bread), typically in the form of loaves or rolls, French baguette-type bread, pita bread, tortillas, cakes, pancakes, biscuits, wafers, cookies, pie crusts, crisp bread, steamed bread, pizza and the like.

The present invention is further described by the following examples that should not be construed as limiting the scope of the invention.

### EXAMPLES

### Enzymes and Origin

The DNA (SEQ ID NO: 1) encoding the PLC of SEQ ID NO: 3 was cloned from a Nectria mariannaeae strain isolated forest soil sample, Jilin province, China.

Phospholipase C from Kionochaeta is described in WO 2012/062817, indicated as SEQ ID NO: 4 herein.

Purifine is a commercial product produced by Verenium/DSM.

In the examples below the phospholipase C enzymes of the present invention are referred to by SEQ ID NO. If the SEQ ID NO contains a signal peptide it is understood that the reference is to the mature sequence of that SEQ ID NO.

### Example 1: Cloning and Expression

The phospholipase encoding gene was cloned by conventional techniques from the strain indicated and inserted into plasmid pCaHj505 (WO 2013/029496). The gene was expressed with the native secretion signal having the following amino acid sequence MQLLSILAVGLGLAQNAFC (amino acid residues 1 to 19 of SEQ ID NO: 3).

Expression in A. oryzae

One clone with the correct recombinant gene sequence was selected and the corresponding plasmid was integrated into the Aspergillus oryzae MT3568 host cell genome. A. oryzae MT3568 is an amdS (acetamidase) disrupted gene derivative of A. oryzae JaL355 (WO 02/40694) in which pyrG auxotrophy was restored by disrupting the A. oryzae acetamidase (amdS) gene with the pyrG gene.

The hydrolytic activity of the phospholipase produced by the Aspergillus transformants was investigated using lecithin/agarose plates (plate assay described in assay section). 20 μl aliquots of the culture broth from the different transformants, or buffer (negative control) were distributed into punched holes with a diameter of 3 mm and incubated for 1 hour at 37° C. The plates were subsequently examined for the presence or absence of a dark violet zone around the holes corresponding to phospholipase activity.

A recombinant Aspergillus oryzae clone containing the integrated expression construct was selected and it was cultivated in 2400 ml of YPM medium (10 g yeast extract, 20 g Bacto-peptone, 20 g maotose, and deionised water to 1000 ml) in shake flasks during 3 days at a temperature of 30° C. under 80 rpm agitation. Culture broth was harvested by filtration using a 0.2 μm filter device. The filtered fermentation broth was used for enzyme characterization.

Expression in A. niger

The 2.1 kb region of Nectria mariannaeae phospholipase C gene was amplified from the plasmid used for A. oryzae transformation and ligated into the pHiTe50 (the plasmid is also described in EP application No: 13181603.5) using BamHI and PmlI restriction sites to create pHiTe127.

Chromosomal insertion into A. niger NN059461 (a derivative of NN059280 which is described in WO 2012/160093) of the phopspholipase C gene with amdS selective marker (pHiTe127) and the empty vector with pyrG marker (pHUda1306) was performed as described in WO 2012/160093. Strains which grew well were purified and subjected to southern blotting analysis to determine the copy number of the gene. Among the strains one with 2-copy phopspholipase C gene was selected.

The phopspholipase C was expressed in shake flask fermentation. Shake flasks containing 100 ml of the seed medium MSS (70 g Sucrose, 100 g Soybean powder (pH 6.0), water to 1 litre) were inoculated with spores from the A. niger strain and incubated at 30° C., with shaking (220 rpm) for 3 days. Ten ml of the seed culture was transferred to shake flasks containing 100 ml of the main medium MU-1 glu (260 g of glucose, 3 g of MgSO₄.7H₂O, 5 g of KH₂PO₄, 6 g of K₂SO₄, amyloglycosidase trace metal solution 0.5 ml and urea 2 g (pH 4.5), water to 1 litre) and incubated at 30° C., with shaking (220 rpm) for 4 days. The culture supernatants were collected by centrifugation and used for sub-sequent purification.

### Example 2: Phospholipase C Purification

### Expressed from A. oryzae

The culture supernatant was precipitated with (NH₄)₂SO₄ followed by dialysis with 20 mm Bis-Tris at pH 6.5. The sample was loaded onto a chromatographic column of Q Sepharose Fast Flow (GE Healthcare) equilibrated with 20 mm Bis-Tris at pH 6.5. A gradient increase of NaCl concentration was applied from zero to 0.35M NaCl in 15 CV (column volume), followed by 0.5M NaCl in 3 CV, and finally to 1M NaCl in 2 CV. The fractions and flowthrough were checked for PLC activity using the p-Nitrophenylphosphorylcholine assay and lecithin plate assay.

Expressed in A. niger

Ammonium acetate (1.8 M) and zinc sulfate (0.5 mM) was added to the culture supernatant, and the pH was adjusted to 7 using HCl or NaOH. Approx. 200 ml bed volume of Toyopearl Butyl 650M (Tosoh) equilibrated with equilibration buffer (10 mM HEPES-NaOH, 1.8 M ammonium acetate, 0.5 mM zinc sulfate, pH 7), was added to approx. 2 L of the supernatant. After incubation at 4° C. for 30 min with stirring, the unbound fraction was removed by filtration on a glass filter. After wash with 200 ml equilibration buffer several times, elution was performed by elution buffer (10 mM HEPES-NaOH, 0.5 mM zinc sulfate, pH 7). The eluted sample was concentrated by PES ultrafiltration membrane Vivacel 250 (Sartorius), and then dialyzed against 10 L of the elution buffer.

### Example 3: N-Terminal Sequence of PLC

N-terminal sequencing analyses were performed using an Applied Biosystems Procise® protein sequencing system. The samples were purified on a Novex® precast 4-20% SDS polyacrylamide gel (Life Technologies). The gel was run according to manufacturer's instructions and blotted to a ProBlott® PVDF membrane (Applied Biosystems). For N-terminal amino acid sequencing the main protein band was cut out and placed in the blotting cartridge of the Procise® protein sequencing system. The N-terminal sequencing was carried out using the method run file for PVDF membrane samples (Pulsed liquid PVDF) according to manufacturer's instructions. The N-terminal amino acid sequence can be deduced from the 7 chromatograms corresponding to amino acid residues 1 to 7 by comparing the retention time of the peaks in the chromatograms to the retention times of the PTH-amino-acids in the standard chromatogram.

The N-terminal sequence of the mature polypeptide (SEQ ID NO: 3) was DWVEDLW corresponding to amino acids residues 37-43. The N-terminal sequence was identical both when expressed in Aspergillus Oryzae and in Aspergillus Niger.

### Example 4: Thermostability of the Phospholipase C

The thermostability of the polypeptide of SEQ ID NO: 3 purified as described in Example 2 was determined by Differential Scanning calorimetry (DSC) using a VP-Capillary Differential Scanning calorimeter (MicroCal Inc., Piscataway, N.J., USA). The thermal denaturation temperature, Td (° C.), was taken as the top of denaturation peak (major endothermic peak) in thermograms (Cp vs. T) obtained after heating enzyme solutions (approx. 0.5 mg/ml) in buffer (50 mM Acetate pH 5.5 with 2 mM ZnSO₄ or 2 mM Ca Cl₂ added) at a constant programmed heating rate of 200 K/hr.

Sample- and reference-solutions (approx. 0.2 ml) were loaded into the calorimeter (reference: buffer without enzyme) from storage conditions at 10 deg C. and thermally pre-equilibrated for 20 minutes at 20° C. prior to DSC scan from 20° C. to 100° C. Denaturation temperatures were determined at an accuracy of approximately +/−1° C.

TABLE 2
Denaturation temperatures

Tm ° C. pH 5.5

| | −ZnSO₄ | +ZnSO₄ |
|---|---|---|
| SEQ ID NO: 3 from A. oryzae | n.d. | 68.5 |
| SEQ ID NO: 3 from A. niger | 71.9 | 69.7 |

n.d. = not determined

### Example 5: Phospholipase C Specificity Towards PC, PE, PI, PA of Purified PLC's

The substrate specificity of the phospholipase C enzymes of the present invention and Purifine were determined using ³¹P-NMR. This assay follows the conversion of individual phospholipids shown in FIG. 1 in an oil environment and reveals the substrate specificity and preference of the phospholipase, and provides an indication of the pH optimum of the enzymes.

### Substrate

A crude soy oil 1 with the following content of the specific phospholipids measured by P-NMR was used.

### PA: 128 ppm Phosphorus (P)

### PE: 141 ppm P

### PI: 103 ppm P

### PC: 157 ppm P

### Unknown: 37 ppm

Other crude oils may also be applied in this assay, e.g. from rapeseed, sunflower, corn, cottonseed, groundnut, ricebran. The primary criteria are that the oil contains minimum 30 ppm of each of the specific phospholipids.

Ensure mixing before the crude oil is pipetted (it precipitates over time). Store at room temperature.

Buffers and Enzyme 0.2 M Cs-EDTA pH 7.5 solution: EDTA (5.85 g) is dispersed in MQ-water (50 mL). The pH is adjusted to 7.5 using 50% w/w CsOH (approx. 30 mL), which will dissolve the EDTA completely. MQ-water is added to a total volume of 100 mL to give a concentration of 0.2 M.

Phosphate standard: 2 mg/mL solution triphenyl phosphate (TPP) in MeOH.

pH buffers:

### 100 mM Na-citrate pH 4.0

### 100 mM Na-citrate pH 5.5

### 100 mM Na-citrate pH 7.0

Enzyme: Dilute to concentrations of 0.9 and 0.09 mg Enzyme Protein (EP)/mL in the three buffers and keep cold to be used the same day.

### Assay

250 micro-L crude oil was weighed into a 2 mL Eppendorf and 25 micro-L enzyme diluted in the desired pH buffer was added (pH, 4.0, 5.5, 7.0, resulting in 10 mg EP/kg oil or 100 mg EP/kg oil). The mixture was incubated in a thermoshaker at 50° C. for 2 h. Then 0.500 mL phosphate standard solution, 0.5 mL Chloroform-d (CDCl₃) and 0.5 mL Cs-EDTA buffer was added. Phase separation was obtained after 30 sek shaking followed by centrifugation (tabletop centrifuge, 3 min, 13,400 rpm). The lower phase was transferred to a NMR-tube. P-NMR with 128 scans, 5 sec delay time was run. The scale reference according to the phosphate internal standard signal (−17.75 ppm) was checked and all signals were integrated. Assignments (approx. ppm at 25° C.): 1.7 (PA), −0.1 (PE), −0.5 (PI), −0.8 (PC). The position of the signals can change significantly according to exact pH value, temperature, sample concentration, etc. The concentration of each species is calculated as “ppm P”, i.e. mg elemental Phosphorus per kg oil sample. Hence, ppm P=I/1(1S)*n(IS)*M(P)/m(oil). % Remaining phospholipid is calculated as the ratio of the phospholipid concentration in the enzyme treated sample to the same concentration in a blank sample.

The results are summarized in tables 3 to 6 below.

TABLE 3
The PC, PE, PI, PA specificity at different pH's
and different enzyme concentrations of phospholipase
C of SEQ ID NO: 3 expressed in A. Niger.

| | % Remaining phospholipid | | | | | | | |
|---|---|---|---|---|---|---|---|---|
| | 100 mg EP/kg oil | | | | 10 mg EP/kg oil | | | |
| | PA | PE | PI | PC | PA | PE | PI | PC |
| pH 4.0 | 68 | 72 | 77 | 68 | 90 | 90 | 90 | 91 |
| pH 5.5 | 26 | 19 | 28 | 33 | 66 | 77 | 60 | 77 |
| pH 7.0 | 10 | 19 | 9 | 30 | n.d. | n.d. | n.d. | n.d. |

n.d = not determined

From these data it can be seen that the PLC of SEQ ID NO: 3 expressed in A. Niger has activity on all four phospholipids with a preferred pH range between 5.5 and 7.0.

TABLE 4
The PC, PE, PI, PA specificity at different pH's
and different enzyme concentrations of phospholipase
C of SEQ ID NO: 3 expressed in A. Oryzae

| | % Remaining phospholipid | | | | | | | |
|---|---|---|---|---|---|---|---|---|
| | 100 mg EP/kg oil | | | | 10 mg EP/kg oil | | | |
| | PA | PE | PI | PC | PA | PE | PI | PC |
| pH 4.0 | n.d. | n.d. | n.d. | n.d. | n.d. | n.d. | n.d. | n.d. |
| pH 5.5 | 17 | 18 | 10 | 16 | 45 | 65 | 41 | 29 |
| pH 7.0 | 7 | 0 | 0 | 11 | 31 | 65 | 23 | 63 |

n.d = not determined

From these data it can be seen that the PLC of SEQ ID NO: 3 expressed in A. oryzae has activity on all four phospholipids with a preferred pH range between 5.5 and 7.0. Especially at the high dosage the PLC of SEQ ID NO: 3 is capable of almost complete PE and PI hydrolysis (below the detection limit) and significant PA and PC hydrolysis at pH 7.0 and significant hydrolysis of all four phospholipids at pH 5.5.

For an activity comparison, the performance of Kionochaeta PLC of SEQ ID NO: 4 and Purifine in oil 1, is shown in table 5 and 6.

TABLE 5
The PC, PE, PI, PA specificity of phospholipase C from Kionochaeta
at different pH's and different enzyme concentrations.

| % Remaining phospholipid | |
|---|---|
| 100 mg EP/kg oil | 10 mg EP/kg oil |

| | PA | PE | PI | PC | PA | PE | PI | PC |
|---|---|---|---|---|---|---|---|---|
| pH 4.0 | 54 | 54 | 83 | 51 | 85 | 91 | 90 | 80 |
| pH 5.5 | 21 | 67 | 51 | 30 | 63 | 83 | 51 | 61 |
| pH 7.0 | 23 | 31 | 13 | 17 | 61 | 78 | 61 | 67 |

From these data it can be seen that the PLC from Kionochaeta has activity on all four phospholipids within the broad pH spectrum from 4.0 to 7.0. However not with as high activity as the PLC of SEQ ID NO: 3 at pH 5.5 and 7.0.

TABLE 6
Phospholipid hydrolysis by Purifine at different
pH's and different enzyme concentrations.

| | % Remaining phospholipid | | | | | | | |
|---|---|---|---|---|---|---|---|---|
| | 100 mg EP/kg oil | | | | 10 mg EP/kg oil | | | |
| | PA | PE | PI | PC | PA | PE | PI | PC |
| pH 4.0 | 107 | 107 | 91 | 114 | 106 | 106 | 98 | 105 |
| pH 5.5 | 107 | 38 | 106 | 20 | 101 | 114 | 106 | 98 |
| pH 7.0 | 89 | 0 | 94 | 0 | 112 | 18 | 102 | 0 |

Purifine is capable of almost complete PC hydrolysis (below the detection limit) and significant PE hydrolysis at pH 7.0. Purifine is also active at pH 5.5 at the high dose.

### Example 6: Degumming Assay

Performance of the phospholipase C enzyme of the present invention (SEQ ID NO: 3) and Kionochaeta PLC (SEQ ID NO: 4) was tested in a degumming assay that mimics industrial scale degumming. The assay measured the following parameters in the oil phase after the degumming:

a) Diglyceride content by High-performance liquid chromatography (HPLC) coupled to Evaporative Light Scattering Detector (ELSD),
b) Quantification of the individual phospholipids species: Phosphatidylcholine (PC); Phosphatidylinositol (PI); Phosphatidylethanolamine (PE); Phosphatidic acid (PA); by Liquid Chromatography quadrupole mass spectrometer time of flight (LC/TOF/MS)
c) Total phosphorus reduction by Inductively coupled plasma optical emission spectrometry (ICP-OES).

The phospholipid composition in the crude soybean oil 2, used in the experiments, is indicated in table 7A. The composition was measured by LC/MS as phosphorus originating from individual phospholipid species.

TABLE 7A
Phospholipid composition of crude oil (mg/kg phosphorus).

| | Crude oil 2 |
|---|---|
| PA | 295 |
| PE | 125 |
| PI | 84 |
| PC | 229 |
| Total | 732 |

The Calcium (Ca), Magnesium (Mg) and Phosphorus (P) composition in the crude soybean oil 2, used in the experiments, is indicated in table 7B. The composition was measured by ICP.

TABLE 7B
Ca, Mg, P composition of crude oil 2 measured by ICP (mg/kg oil)

| Ca | Mg | P |
|---|---|---|
| 86 | 64 | 709 |

### Degumming Assay

Crude soybean oil (75 g) was initially acid/base pretreated (or not) to facilitate conversion of insoluble phospholipids salt into more hydratable forms and ensure an environment suitable for the enzyme. Acid/base pretreatment was done by acid addition of Ortho Phosphoric acid (85% solution) applied in amounts equal to 0.05% (100% pure Ortho Phosphoric acid) based on oil amount and mixing in ultrasonic bath (BRANSON 3510) for 5 min and incubation in rotator for 15 min followed by base neutralization with 4 M NaOH applied in equivalents (from 0.5 to 1.5) to pure Ortho Phosphoric acid in ultrasonic bath for 5 min. The enzyme reaction was conducted in low aqueous system (3% water total based on oil amount) in 100 ml centrifuge tubes, cylindrical, conical bottom. Samples were ultrasonic treated for 5 min, followed by incubation in a heated cabinet at selected temperature (from 50 to 60° C.) with stirring at 20 rpm for a selected incubation time (from 1 to 5 hours). To separate the mixture into an oil phase and a heavy water/gum phase the samples were centrifuged at 700 g at 85° C. for 15 min (Koehler Instruments, K600X2 oil centrifuge).

### a) Diglyceride Measurement

The HPLC-ELSD method (using DIONEX equipment and Lichrocart Si-60, 5 μm, Lichrosphere 250-4 mm, MERCK column) was based on the principle of the AOCS Official Method Cd 11 d-96 and quantifies the diglyceride content down to 0.1 wt %.

### b) Quantitative Analysis of Phospholipids by LCMS/MS

Liquid Chromatography coupled to triple quadrupole mass spectrometer (LC/MS/MS) or coupled to quadrupole mass spectrometer time of flight (LC/TOF/MS) was used to quantify the individual phospholipids species: phosphatidylcholine (PC); Phosphatidylinositol (PI); Phosphatidylethanolamine (PE) and Phosphatidic acid (phosphatidate) (PA). The sensitivity of the assay goes down to less than 1 mg Phosphorus/kg oil for PC, PE and PI (ppm) and less than 10 mg Phosphorus/kg for PA. The oil sample was dissolved in chloroform. The extract was then analysed on LC-TOF-MS (or on LC-MS/MS if lower detection limits are needed) using following settings:

### LC-Settings

Eluent A: 50% Acetonitril, 50% Water, 0.15% formic acid
Eluent B: 100% Isopropionic acid, 0.15% formic acid
Run time: 26.9 min
Flow: 0.50 mL/min
Column temperature: 50° C.
Autosampler temp: 15-25° C.
Injection volume: 1 μL
Column type Material: Charged Surface Hybrid, length: 50 mm, size: 1.7 μm, ID: 2.1 mm

### MS-Settings

| TOF/MS | MS/MS (Xevo) |
|---|---|
| Capillary: 3.50 kV | Capillary: +3.50/−2.0 kV |
| Cone: 28 | Cone: Component specific |
| Extractor: 2 V | Extractor: 2.5 V |
| RF-lens: 0.5 V | RF-lens: |
| Source temp: 125° C. | Source temp: 150° C. |
| Desolvation temp: 500° C. | Desolvation temp: 500° C. |
| Cone gas flow: 30 L/hour | Cone gas flow: 30 L/hour |
| Desolvation gas flow: 850 L/hour | Desolvation gas flow: 850 L/hour |

The data was processed using MassLynx version 4.1 Software. In the below examples the method is just termed LCMS.

### c) Phosphorus/Phospholipid Measurement

The ICP-OES quantifies the phosphorus (P) content and other metals such as Ca, Mg, Zn down to 4 ppm with an accuracy of approximately ±1 ppm P.

Example 7 to 9 below describes results obtained using the degumming assay of this example.

### Example 7: Enzyme Robustness to Different Acid/Base Pre-Treatments of Oil at 50° C.

The PLC enzyme of SEQ ID NO: 3 expressed in A. oryzae was applied in the degumming assay testing different acid/base pre-treatments of crude oil 2. The applied enzyme concentration was 5 mg EP/kg oil. The increase in diglyceride content after 3, 5 and 22 hours measured by HPLC-ELSD as well as the calcium, magnesium and phosphorus content after 22 hours enzyme incubation measured by ICP is presented in Table 8.

TABLE 8
Diglyceride increase (% w/w) and Ca, Mg and P content after
enzyme treatment of acid/base treated crude soybean oil 2.

| | Diglyceride increase | | | Metal content | | |
|---|---|---|---|---|---|---|
| | (% w/w) | | | after 22 h | | |
| Acid/base pre-treatment | 3 h | 5 h | 22 h | Ca | Mg | P |
| 0.05% PA + 0.5 eqv NaOH | 0.20 | 0.23 | 0.58 | 50 | 12 | 12 |
| 0.05% PA + 1.0 eqv NaOH | 0.13 | 0.23 | 0.74 | 38.5 | 9 | 29 |
| 0.05% PA + 1.5 eqv NaOH | 0.19 | 0.34 | 0.76 | 50 | 13 | 43 |
| No acid/base | 0.05 | 0.12 | 0.54 | 35 | 8 | 45 |

In the degumming assay the PLC enzyme of SEQ ID NO: 3 resulted in a significant diglyceride formation at 50° C. The enzyme showed good performance under all acid/base assisted degumming conditions and reasonable performance in water degumming (no acid/base) after 22 hours treatment. The Phosphorus content of the oil was reduced by the degumming treatment.

### Example 8: Degumming at 50° C.

The PLC enzyme of SEQ ID NO: 3 expressed in A. oryzae was applied in the degumming assay at two different concentrations and at two different acid/base pre-treatment conditions. The degumming performance was compared to the Kionochaeta PLC of SEQ ID NO: 4. The diglyceride increase after enzymatic degumming for 3, 5 and 24 hours was measured by HPLC-ELSD. Calcium, magnesium and phosphorus content was measured by ICP after 24 hours. The results are shown in Table 9.

TABLE 9
Diglyceride increase (% w/w) and Ca, Mg, P content (ppm) after
enzyme treatment of acid/base treated crude soybean oil.

| | | Enz. | | | | | | |
|---|---|---|---|---|---|---|---|---|
| | | Dosage | Enzyme incubation | | | | | |
| Acid/base | | (mg EP/ | (hours) | | | | | |
| pre-treatment | Enzyme | kg oil) | 3 | 5 | 24 | Ca | Mg | P |
| 0.05% PA + | SEQ ID | 4.75 | 0.10 | 0.14 | 0.46 | 15 | <5 | 17 |
| 0.5 eqv | NO: 3 | | | | | | | |
| NaOH | | | | | | | | |
| 0.05% PA + | SEQ ID | 8.5 | 0.17 | 0.27 | 0.80 | 8 | <5 | 12 |
| 0.5 eqv | NO: 3 | | | | | | | |
| NaOH | | | | | | | | |
| 0.05% PA + | Kionochaeta | 16 | 0.00 | 0.09 | 0.37 | 19 | <5 | 21 |
| 0.5 eqv | PLC | | | | | | | |
| NaOH | | | | | | | | |
| 0.05% PA + | SEQ ID | 4.75 | 0.28 | 0.38 | 0.95 | 36 | 10 | 47 |
| 1.5eqv | NO: 3 | | | | | | | |
| NaOH | | | | | | | | |
| 0.05% PA + | SEQ ID | 8.5 | 0.44 | 0.59 | 1.20 | 28 | 6 | 31 |
| 1.5eqv | NO: 3 | | | | | | | |
| NaOH | | | | | | | | |
| 0.05% PA + | Kionochaeta | 16 | 0.03 | 0.13 | 0.88 | 41 | 12 | 58 |
| 1.5eqv | PLC | | | | | | | |
| NaOH | | | | | | | | |

The highest measured diglyceride increase after 24 h (1.20% w/w) corresponds to roughly 70% of the maximum theoretical DG formation, calculated based on 709 ppm total P corresponding to 1.74% w/w phospholipid with average MW 761 g/mol. In terms of DG formation and phosphorus reduction the degumming performance of the PLC enzyme of SEQ ID NO: 3 was superior compared to the Kionochaeta PLC under both tested acid/base treatment conditions. In particular the PLC of the present invention appeared to form diglyceride quicker than the Kionochaeta PLC.

### Example 9: Dosis Response in Degumming at 60° C.

The PLC enzyme of SEQ ID NO: 3 expressed in A. Niger was applied in the degumming assay at 60° C. The crude oil 2 was pre-treated with 0.05% phosphoric acid and 1.5 molar equivalents of NaOH prior to incubation with enzymes. The diglyceride increase after enzymatic degumming for 2, 3 and 5 hours was measured by HPLC-ELSD. The results are shown in Table 10.

TABLE 10
Diglyceride increase (% w/w) after enzyme treatment in different doses

| | Enzyme Incubation | | |
|---|---|---|---|
| | time (hours) | | |
| Enzyme dosis (mg EP/kg Oil) | 2 | 3 | 5 |
| 4 | 0.06 | 0.08 | 0.12 |
| 10 | 0.09 | 0.14 | 0.20 |
| 20 | 0.23 | 0.31 | 0.42 |
| 50 | 0.32 | 0.39 | 0.50 |

Clear dosis response effects were observed and increased diglyceride formation at prolonged enzyme incubation time.

### Example 10: Degumming at 50° C.

The PLC enzyme of SEQ ID NO: 3 expressed in A. niger was applied in the degumming assay at 50° C. and compared to the fungal Kionochaeta PLC of SEQ ID NO: 4 and the bacterial Purifine. The crude oil 2 was pre-treated with 0.05% phosphoric acid and 1.5 molar equivalents of NaOH prior to incubation with enzymes. The diglyceride increase after enzymatic degumming for 3, 5 and 24 hours was measured by HPLC-ELSD as well as the calcium, magnesium and phosphorous content measured by ICP after 24 hours. The results are shown in Table 11.

TABLE 11
Diglyceride increase (% w/w) after enzyme treatment of acid/base
treated crude soybean oil, measured by HPLC-ELSD.

| Temp ° C. | Enzyme | Enz. Dosage | 3 h | 5 h | 24 h |
|---|---|---|---|---|---|
| 50 | Purifine PLC | 200 ppm | 0.37 | 0.39 | 0.50 |
| 50 | Kionochaeta PLC | 10 mg EP/kg oil | 0.11 | 0.18 | 0.72 |
| 50 | SEQ ID NO: 3 | 10 mg EP/kg oil | 0.20 | 0.27 | 0.61 |

In terms of diglyceride formation the degumming performance of the PLC enzyme of SEQ ID NO: 3 was superior compared to the Kionochaeta PLC at 3 and 5 hours. Due to the broad specificity both fungal PLC's were superior over Purifine PLC which only removes PC and PE if the reaction was allowed to run for 24 hours.

### Example 11: Degumming at 60° C., 3% or 5% Water Content

The PLC enzyme of SEQ ID NO: 3 expressed in A. niger was applied in the degumming assay at two different water contents. The crude oil 2 was pre-treated with 0.05% phosphoric acid and 1.5 molar equivalents of NaOH prior to incubation with enzymes. The degumming performance was compared to the Kionochaeta PLC of SEQ ID NO: 4. The diglyceride increase after enzymatic degumming for 2, 3 and 24 hours was measured by HPLC-ELSD. Calcium, magnesium and phosphorus content was measured by ICP after 24 hours. The results are shown in Table 12.

TABLE 12
Diglyceride increase (% w/w) and Ca, Mg, P content (ppm) after
enzyme treatment of acid/base treated crude soybean oil.

| | | Enz. | | | | | | |
|---|---|---|---|---|---|---|---|---|
| Water | | Dosage | | | | | | |
| content, | | (mg EP/ | DG increase, % w/w | | | | | |
| % w/w | Enzyme | kg oil) | 2 h | 3 h | 24 h | Ca | Mg | P |
| 3 | None (Blank) | 0 | 0.12 | 0.16 | 0.19 | 55 | 24 | 95 |
| 3 | SEQ ID | 10 | 0.32 | 0.42 | 1.24 | 29 | 7 | 40 |
| | NO: 3 | | | | | | | |
| 3 | None (Blank) | 0 | 0.12 | 0.16 | 0.19 | 55 | 24 | 95 |
| 3 | Kionochaeta | 10 | 0.25 | 0.33 | 1.85 | 30 | 10 | 45 |
| | PLC | | | | | | | |
| 5 | None (Blank) | 0 | 0.13 | 0.13 | 0.11 | 24 | 8 | 38 |
| 5 | SEQ ID | 10 | 0.51 | 0.60 | 1.53 | 23 | 5 | 26 |
| | NO: 3 | | | | | | | |
| 5 | Kionochaeta | 10 | 0.30 | 0.36 | 1.50 | 27 | 5 | 38 |
| | PLC | | | | | | | |

It was observed that the PLC enzyme of SEQ ID NO: 3 formed diglycerides quicker than the Kionochaeta PLC. resulting in higher DG levels after 2 h and 3 h reaction time at both tested water contents. In terms of phosphorus reduction the degumming performance of the PLC enzyme of SEQ ID NO: 3 was also superior compared to the Kionochaeta PLC under both tested water contents.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A polypeptide having phospholipase C activity, selected from the group consisting of:
a. a polypeptide having at least 70% sequence identity to the mature polypeptide of SEQ ID NO: 3;
b. a polypeptide encoded by a polynucleotide that hybridizes under low stringency conditions with (i) the mature polypeptide coding sequence of SEQ ID NO: 1, ii) the cDNA sequence thereof or iii) the full-length complement of i) or ii);
c. a polypeptide encoded by a polynucleotide having at least 70% sequence identity to the mature polypeptide coding sequence of SEQ ID NO: 1 or the cDNA sequence thereof;
d. a variant of the mature polypeptide of SEQ ID NO: 3 comprising a substitution, deletion, and/or insertion at one or more positions; and
e. a fragment of the polypeptide of (a), (b), (c), or (d) that has phospholipase C activity.

2. The polypeptide of claim 1, comprising or consisting of or consisting essentially of SEQ ID NO: 3 or the mature polypeptide of SEQ ID NO: 3 or amino acids 20 to 632 of SEQ ID NO: 3 or amino acids 37 to 632 SEQ ID NO: 3.

3. The polypeptide of claim 1, wherein the polypeptide has activity towards all of the following phospholipids, phosphatidic acid (PA), phosphatidylcholine (PC), phosphatidylethanolamine (PE) and phosphatidyl inositol (PI).

4. The polypeptide of claim 1, wherein the fragment is more than 550 amino acids and the fragment has phospholipase C activity.

5. A composition comprising the polypeptide of claim 1.

6. A polynucleotide encoding a polypeptide according to claim 1.

7. A nucleic acid construct or expression vector comprising the polynucleotide of claim 6 operably linked to one or more control sequences that direct the production of the polypeptide in an expression host.

8. A recombinant host cell comprising the polynucleotide of claim 6 operably linked to one or more control sequences that direct the production of the polypeptide.

9. A method of producing a polypeptide of claim 1, comprising cultivating a cell, which in its wild-type form produces the polypeptide, under conditions conducive for production of the polypeptide.

10. A method of producing a polypeptide of claim 1, comprising cultivating the host cell of claim 8 under conditions conducive for production of the polypeptide.

11. The method of claim 9, further comprising recovering the polypeptide.

12. (canceled)

13. A method for reducing the content of phospholipids in an oil composition, comprising
a. contacting said oil with the polypeptide of claim 1, under conditions sufficient for the enzyme to react with the phospholipids to create diglyceride and phosphate ester and/or phosphoric acid, and;
b. separating the phosphate ester from the oil composition.

14. The method according to claim 13, wherein the oil is an edible oil.

15. The method according to claim 13, wherein the oil is selected from crude oil, water degummed oil, caustic refined oil and acid degummed oil.

16. The method according to claim 13, where in the oil comprises phosphatidic acid (PA), phosphatidylcholine (PC), phosphatidylethanolamine (PE) and phosphatidyl inositol (PI).


SEQUENCE LISTING (4 sequences)

SEQ ID NO: 1 - DNA, 2115 bp, Nectria mariannaeae
  sig_peptide (1)..(57)
  Intron (169)..(222)
  Intron (310)..(360)
  Intron (666)..(727)
  Intron (937)..(985)
atgcaattactctctatcctcgccgtcgggctcggcctcgcgcagaacgcattctgccaagaggtcactcatgatcttgccggtatcaagcgatccctagaatccagagattgggttgaggatctttgggataagttcgaaagtgatgcaacatgcgcgggatgcgaggttagccattcatgttgatttggtgttgagcatgacgagctaatgaaatattagtcactcgtgttagtgctcaagggcttggccgctataagtgatcaagcgtttattgacgtccttcaggagatttgcaagatctccggggtgagtacaccttgactatggtgcttctaagtggagttgacgatgcagtaggctgaggatgacgatgtttgtgacggttcaattcaacttgaaggccccgttattgccagcggtcttcgatcaatggctatcggctctcgaacttccaaagaattctgtactacattccttggactttgcgcgtaccctgcggtgcagcaatggagtgttcccttctcgtcctccaagtcttccaagactcgtccttcgtccagtggaaaggaccctatcaaggtggttcattattctgatattcatatcgatcctttatatgtcgggggctcaaactccaactgcactaagcccatatgttgtaggtaagagataaccccaaacacttgccagcaaaactggacatgttgataactaaactacgaaggtcatacaccaaggctgaccagcccgggaacaacaaatatcctgctggaccgaacggcgaccataactgcgattctccagtcagtcttgagaagagcatgtacaacgccatcaaggaaatcgttccagatgcagccttcaccatcttcaccggggacattgtcgatcatgcagtctggaatactagccaatcctataatacggaacaaagtgtgttcagtataatccatccagtggaataacttactaaatctcacagttaccaatgcctacggcttgatgagtgataatctgggcacaatctatgggactgccggcaaccatgaagctcatcccgctaatgccttccaacctaactccgtcggcaacgtgagccaatgggtatacgatcttctttcaggtctctggtcacagtggattagcaccgaagccaaagctgactcggagaagcttggcgcttactctaccaagtatcctggcggcaacttgcgcattatctctctcaacaccaacatgtactatcgagaaaactactggctctaccgcaagacgatgattcaggaccctagcaatcagatttcctggctcgtaaacgaactcgaagccgctgagactgcgggcgagcgcgtttatatcatcggccacatgccgcttggagactctaacagttttcacgaccagtccaactaccttgaccaagtaatcaaccgctactccgcaactatctctgccatgttctttggccacacacacgacgaccagttccagataagttactccaattggtcaaatcgcaacttctccaatgccctcgtaacctcctacattggtccctcactcactcccaccgccggtatgcccgcctttcgcgtttacgatgtcgaccccgtgacgttcggaatcctcgactcgaccacatacatcgctgacatgaccgactctgcctttcaaaccactggcccagtgtggaagaagtattattctgccaaagaagtttacggctctttattgagcccggctgtgactgacagcagcgccgagctcacggcagccttctggcacaacgtgacgaccctgttcgaggcggacaacaccgcatttgaggcgtttctttcccgcaagagccgtgggtggaagtcagaatcctgcacaggcacttgcaaggccaacgagatctgtcaattgcgcgcggctcgcagcgagaacaattgctacaccccgtcgctaggaattagcttcaacaaacgaagtctgaacccagttgaagagcgggacgagtgtgggatttcagtgactagggctacggttagtgctatgggcgtaaggaaagacgttttgcgtttgttaaagaagaggtttatcgagaaggcgggtgaggtccgtggctga

SEQ ID NO: 2 - DNA, 1899 bp, Artificial Sequence
  cDNA
atgcaattactctctatcctcgccgtcgggctcggcctcgcgcagaacgcattctgccaagaggtcactcatgatcttgccggtatcaagcgatccctagaatccagagattgggttgaggatctttgggataagttcgaaagtgatgcaacatgcgcgggatgcgagtcactcgtgttagtgctcaagggcttggccgctataagtgatcaagcgtttattgacgtccttcaggagatttgcaagatctccggggctgaggatgacgatgtttgtgacggttcaattcaacttgaaggccccgttattgccagcggtcttcgatcaatggctatcggctctcgaacttccaaagaattctgtactacattccttggactttgcgcgtaccctgcggtgcagcaatggagtgttcccttctcgtcctccaagtcttccaagactcgtccttcgtccagtggaaaggaccctatcaaggtggttcattattctgatattcatatcgatcctttatatgtcgggggctcaaactccaactgcactaagcccatatgttgtaggtcatacaccaaggctgaccagcccgggaacaacaaatatcctgctggaccgaacggcgaccataactgcgattctccagtcagtcttgagaagagcatgtacaacgccatcaaggaaatcgttccagatgcagccttcaccatcttcaccggggacattgtcgatcatgcagtctggaatactagccaatcctataatacggaacaaattaccaatgcctacggcttgatgagtgataatctgggcacaatctatgggactgccggcaaccatgaagctcatcccgctaatgccttccaacctaactccgtcggcaacgtgagccaatgggtatacgatcttctttcaggtctctggtcacagtggattagcaccgaagccaaagctgactcggagaagcttggcgcttactctaccaagtatcctggcggcaacttgcgcattatctctctcaacaccaacatgtactatcgagaaaactactggctctaccgcaagacgatgattcaggaccctagcaatcagatttcctggctcgtaaacgaactcgaagccgctgagactgcgggcgagcgcgtttatatcatcggccacatgccgcttggagactctaacagttttcacgaccagtccaactaccttgaccaagtaatcaaccgctactccgcaactatctctgccatgttctttggccacacacacgacgaccagttccagataagttactccaattggtcaaatcgcaacttctccaatgccctcgtaacctcctacattggtccctcactcactcccaccgccggtatgcccgcctttcgcgtttacgatgtcgaccccgtgacgttcggaatcctcgactcgaccacatacatcgctgacatgaccgactctgcctttcaaaccactggcccagtgtggaagaagtattattctgccaaagaagtttacggctctttattgagcccggctgtgactgacagcagcgccgagctcacggcagccttctggcacaacgtgacgaccctgttcgaggcggacaacaccgcatttgaggcgtttctttcccgcaagagccgtgggtggaagtcagaatcctgcacaggcacttgcaaggccaacgagatctgtcaattgcgcgcggctcgcagcgagaacaattgctacaccccgtcgctaggaattagcttcaacaaacgaagtctgaacccagttgaagagcgggacgagtgtgggatttcagtgactagggctacggttagtgctatgggcgtaaggaaagacgttttgcgtttgttaaagaagaggtttatcgagaaggcgggtgaggtccgtggctga

SEQ ID NO: 3 - protein, 632 aa, Nectria mariannaeae
  SIGNAL (1)..(19)
  PROPEP (20)..(36)
MQLLSILAVGLGLAQNAFCQEVTHDLAGIKRSLESRDWVEDLWDKFESDATCAGCESLVLVLKGLAAISDQAFIDVLQEICKISGAEDDDVCDGSIQLEGPVIASGLRSMAIGSRTSKEFCTTFLGLCAYPAVQQWSVPFSSSKSSKTRPSSSGKDPIKVVHYSDIHIDPLYVGGSNSNCTKPICCRSYTKADQPGNNKYPAGPNGDHNCDSPVSLEKSMYNAIKEIVPDAAFTIFTGDIVDHAVWNTSQSYNTEQITNAYGLMSDNLGTIYGTAGNHEAHPANAFQPNSVGNVSQWVYDLLSGLWSQWISTEAKADSEKLGAYSTKYPGGNLRIISLNTNMYYRENYWLYRKTMIQDPSNQISWLVNELEAAETAGERVYIIGHMPLGDSNSFHDQSNYLDQVINRYSATISAMFFGHTHDDQFQISYSNWSNRNFSNALVTSYIGPSLTPTAGMPAFRVYDVDPVTFGILDSTTYIADMTDSAFQTTGPVWKKYYSAKEVYGSLLSPAVTDSSAELTAAFWHNVTTLFEADNTAFEAFLSRKSRGWKSESCTGTCKANEICQLRAARSENNCYTPSLGISFNKRSLNPVEERDECGISVTRATVSAMGVRKDVLRLLKKRFIEKAGEVRG

SEQ ID NO: 4 - protein, 643 aa, Kionochaeta sp
  chain (19)..(625)
MRASSILSLALGLSVAQAAVNPADVLSVVEKRVDPASGLEVRSIWDTIWNDIKSAADCTACEAVLTLLKGVAAFGDNFFVEVLTEICDLSGAEDDDVCSGVLSLEGPIIANDIRKMSIGSKTSELFCITFLGLCSYPAVDAFTVPFPTAKSAATRPVSSGKDPIYVVHYSDIHIDPFYVAGSASNCTKPICCRDYTSASSPGNNNSPAGPYGDHNCDVPISLEDSMYAAIKKLVPDAAFGIFTGDIVDHAVWNTSESQNIIDMNDAYTRMKNSGMLPTIFATAGNHEASPVNSFPPPAIGNESQWVYDTLASDWSQWIGTSGASSVESIGAYSVQYGSTKLRVISLNTNMYYIENFYLYEPTMEQDPAGQFAWLVSELSAAEAAGERVWIIGHMPLGLSDAFHDPSNYFDQIVNRYEATIAAMFFGHTHEDHFQISYSDYNARTAANARAVSYIMPSLTPTSGHPTFRVYTVDPETFGVLDATTYYADMSQPTYQTAGPAWSVYYSAKAAYGGLVDPPVAADDAAAELTPAFWHNVTAALAADPASFDAYYARKTRGWDVAACAGACAAAEVCALRAARAQDNCVVPTPGVHFSKRADEGTLAHHRDECGVSVARNSLSSLVVQREALEHLEGRLSEKRRMAV